(19) United States
(12) Patent Application Publication
(10) Pub. No.: US 2010/0286598 A1
(43) Pub. Date: Nov. 11, 2010

(54) HYPODERMIC NEEDLE WITH WATER-SOLUBLE OBSTRUCTION FOR THE ADMINISTRATION OF DRUGS AND VACCINES

(76) Inventor: Arcadio Garcia De Castro Andrews, Hoyo De Manzanares (ES)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/682,209

(22) PCT Filed: Oct. 8, 2008
(86) PCT No.: PCT/EP08/63494

§ 371 Date: Jul. 27, 2010

(30) Foreign Application Priority Data
Oct. 9, 2007 (ES) P200702650

(51) Int. Cl. A61M 5/32

(52) U.S. Cl. 604/60; 604/192

(57) ABSTRACT

The application refers to hypodermic needles (101) for the parenteral administration of liquids. The needles incorporate at their sharp-end an obstruction (102) of rapidly water-soluble material that dissolves within living tissues once the needle is injected. These needles ensure that the end user can only operate the syringe, or injection device, once the needle has been injected. The use of these hypodermic needles avoids the possibility of the user expelling any amount of liquid prior injection, ensuring the administration of the complete dose. These hypodermic needles are of special interest in preloaded injection devices and in the administration of small volumes of liquid were ejection of part of the dose by the user may result in the administration of only a fraction of the intended dose.

### INVENTION FIELD

The invention refers to hypodermic needles for the parenteral administration of liquids and medicines. The hypodermic needles incorporate a simple addition that ensures that the user cannot expel any amount of the liquid before injection of the needle into the receiving animal or human.

### BACKGROUND

Many drugs are administered parenterally by means of injection by trained medical personnel. The misconception that the air container in the needle can become a problem upon injection has lead to the widespread custom of eliminating the air from the needle by means of a gentle pressure over the syringe until a small amount of liquid is expelled through the sharp end of the needle. This does not generally represent a problem in the administration of large volumes where the potential loss represents a small fraction of the total volume of liquid to be injected. However, this practice can become a serious limitation in the injection of small volumes of liquids or in situations in which the first fractions of liquid to be injected contains a large fraction of the dose of the drug to be administered.

Un example of preloaded devices that incorporate small volumes for the low cost administration of medicines is exemplified by Uniject™ Devices of this kind have also been proposed for the administration of formulations that do not require refrigeration. Examples of these formulations are described in patent application WO-98/41188 that incorporates solid suspensions in injectable oils, and also in patent application WO-02/032402 that incorporates fluorocarbons as a means to suspend solid soluble particles containing a drug or vaccine. However, in these preloaded devices incorporating small volumes, the common practice of expelling the air contained in the needle can result in the loss of a considerable fraction of the dose to be administered.

Another method proposed in the administration of drugs and vaccines incorporates the stabilisation of drugs in a solid form in a cartridge located at the syringe end of the hypodermic needle. Devices of this kind are an object of an innovation program promoted by the World Health Organisation [Lloyd J. (2000) Technologies for Vaccine Delivery in the 21st Century. WHO/V&B/00.35]. Examples of these devices are described in patent application US200310068354 that incorporates a membrane on which the DNA material which constitutes the vaccine is lyophilised, and in patent application WO2007/057717 which incorporates a cartridge holding a fibrillar support with a large surface area on which the drug or vaccine is stabilised within a fine film of dry amorphous glass.

In both cases, the injection of an aqueous solution through the cartridge results in the rapid dissolution and carry over of the medicament, most of it in the first fraction of the liquid. Once more, the ejection of a small amount of liquid prior to injection can result in the administration of a suboptimal dose of the drug or vaccine.

In general, the existing devices for the parenteral administration of liquids do not provide a method to avoid the user in expelling a small amount of liquid through the injection needle prior to the injection, and this can originate a considerable reduction of the dose to be administered.

It would therefore be desirable to have simple means in the parenteral administration of liquids that prevents the end user from expelling a fraction of the medicament prior to injection. This would be of particular interest in the administration of non-aqueous suspensions of medicines, or in the administration of medicines which are stabilised within a cartridge at the syringe-end of the needle, were the initial disposal of a small volume may carry-over a considerable proportion of the total dose to be administered.

### INVENTION SUMMARY

The common custom of sanitary personnel to expel an amount of liquid before proceeding to the injection of a drug or medicament can result in the administration of a fraction of the recommended dose. This is particularly relevant in the parenteral administration of small volumes, in preloaded devices, or in devices that incorporate the drug or vaccine in a cartridge in-line within the syringe-end of the needle. The incorporation of a soluble obstruction within the sharp-end of the hypodermic needle provides needles that are only operational once they are injected and the soluble obstruction dissolves in the tissues of the injected human or animal subject. These needles avoid the possibility that the user can expel part of the dose prior to injection and facilitate the administration of the full dose of the drug or vaccine.

### DESCRIPTION OF THE FIGURES

FIGS. 1A-D. Represent the steps in the process of injection of a hypodermic needle which contains a soluble obstruction at its sharp-end. FIGS. 1A-D represent longitudinal sections of the different moments in the injection process of a hypodermic needle 101 that incorporates in its sharp-end an obstruction 102 with a soluble material. FIG. 1A represents a hypodermic needle prior being used. FIG. 1B represents a hypodermic needle at the moment of injection in the human or animal tissues 105. FIG. 1C represents a hypodermic needle at the moment immediately alter injection once the obstruction 102 has dissolved trough contact with the surrounding tissues. FIG. 1D represents the hypodermic needle at the moment in which the drug or vaccine in liquid solution or suspension 104 is administered.

FIGS. 2A-B. Represent a preloaded device loaded with a drug or vaccine in a liquid format and incorporating a soluble obstruction at the sharp-end of the hypodermic needle. FIG. 2A represents a longitudinal section of an injector device 203 for drugs and vaccines in a liquid solution or suspension 204 that incorporate a soluble obstruction 202 at the sharp-end of a hypodermic needle 201. FIG. 2B represents an amplified view of the sharp-end of the hypodermic needle 201 and the soluble obstruction 202.

FIGS. 3A-B. Represent a device containing a drug or vaccine in a solid format within a cartridge of a hypodermic needle and containing a soluble obstruction at the sharp-end of the hypodermic needle.

FIG. 3A represents a longitudinal section of a device that at the sharp-end of a hypodermic needle 301 incorporates a soluble obstruction 302. The drug 306 in a solid format is incorporated within a cartridge 305 located at the syringe end of the hypodermic needle 301. The injection device 303 contains water for injection 304. FIG. 3B represents an amplified view of the sharp-end of the hypodermic needle 301 and the soluble obstruction 302.

### DETAILED DESCRIPTION OF THE INVENTION

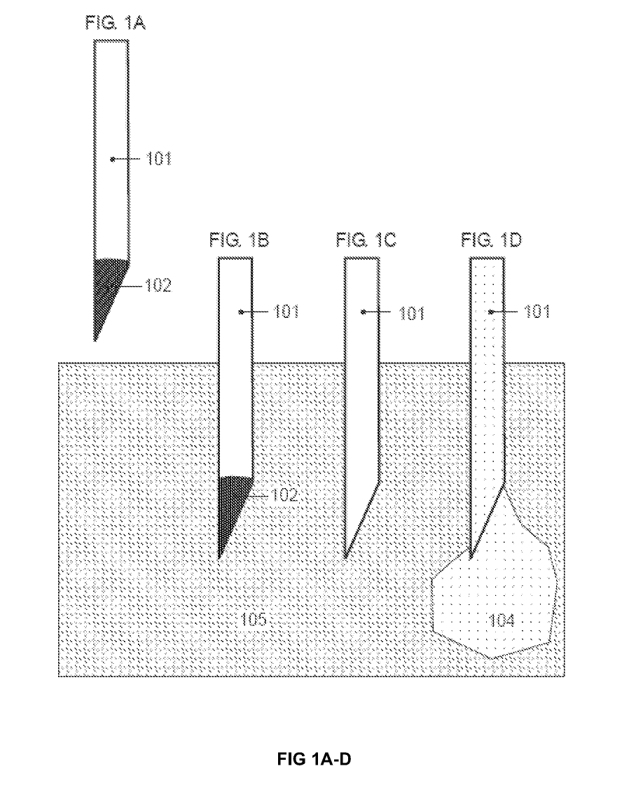

The present invention refers to hypodermic needles 101, 201 or 301 for the administration of solutions and suspensions of drugs, medicines and vaccines, that contain at their sharp-end a water-soluble obstruction 102, 202 or 302, as is represented in FIGS. 1A-D, 2A-B and 3A-B.

Penetration of the hypodermic needles 101, 201 or 301 in living tissue of a human or animal subject results in the almost instantaneous dissolution of the obstruction 102, 202 or 203, and permits the injection of the liquid containing the drug or vaccine. The incorporation of the obstruction 102, 202 or 302 avoids the user from expelling part of the liquid contained within the injection device prior to the needle penetrating the tissues of the receiving animal or human subject. The hypodermic needles 101, 201 or 301 have preferred internal diameters ranking from 0.1 mm y 1 mm, and the capacity of administering drugs and vaccines by injection to depths up and over 5 cm. However it is acknowledged that larger internal diameter needles may be applicable in other fields of animal and plant health. The obstruction 102, 202 or 302 is preferably composed by materials which are rapidly soluble in aqueous media and that dissolve once the needle penetrates living tissues. Examples of these include, with out limitation, carbohydrates, sugar alcohols, amino acids, proteins, polymers, salts and/or mixtures thereof. Applicable carbohydrates and sugar alcohols include, with out limitation, maltodextrin, trehalose, raffinose, cellobiose, melezitose, glucose, fructose, maltulose, iso-maltulose, lactulose, maltose, gentobiose, lactose, galactose, isomaltose, manose, maltitol, lactitol, eritrol, palatinitol, inositol, xilitol, mannitol, sorbitol, dulcitol and/or ribitol and mixtures thereof. Preferably the carbohydrates are chosen from those that are acceptable for injection, with great solubility, and solid at a temperature of at least 45° C., like for example mannitol, trehalose, raffinose, sucrose and/or glucose. Preferably the amino acids are chosen among those that are acceptable for injection, highly soluble and solid at a temperature up to 45° C. like for example proline, cysteine, arginine, glutamine and/or glycine, and mixtures thereof. Applicable proteins include, with out limitation, collagen, hyaluronic acid, fibronectin, gelatine, and/or agaroses, and mixtures thereof. The applicable polymers, include with out limitation, polyvinyl alcohols, polyvinylpyrrolidone and/or polyethylene glycols, and mixtures thereof. Compatible solutes such as, with out limitation, glycine-betaine, ectoin, hydroxyectoin, can also be incorporated. Moreover, the obstruction 102, 202 or 302 can incorporate materials that result in an effervescent reaction when in contact with an aqueous media. Examples of this, with out limitation, include sodium carbonates. Other acceptable salts which are rapidly soluble and have the additional benefit of being efflorescent include, sodium sulphate, acetate trihydrate, tetraborate decahydrate (Borax), bromoiridite dodecahydrate, carbonate heptahydrate metaperiodate trihydrate, metaphosphate hexahydrate, hydrogen orthophosphate dodecahydrate, sulphite heptahydrate, thiosulphate pentahydrate, calcium lactate, magnesium salicylate tetrahydrate, magnesium sulphate heptahydrate, and ammonium sulphate.

The incorporation of the obstruction 102, 202 or 302 in the hypodermic needle can be done by means of the incorporation of solutions of the soluble material and subsequent solidification by evaporation. The obstruction can also be incorporated by heating of the solid obstruction materials at temperatures in which they become malleable or liquid. The incorporation of a predetermined volume and subsequent cooling results in the fabrication of the desired soluble obstruction. Other methods that permit the incorporation of a soluble obstruction include the incorporation of soluble membranes or puncture with the needle of a solid matrix that becomes incorporated at the needle end.

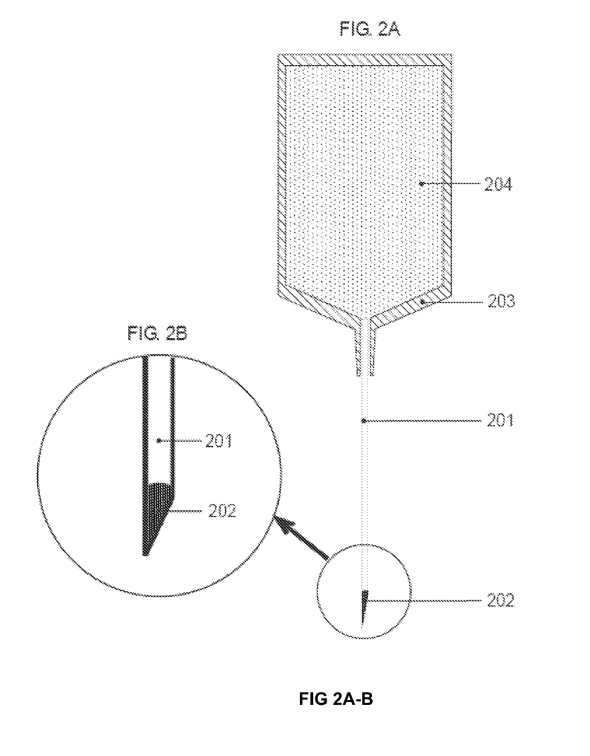

The present invention is applicable, among others, to preloaded devices such as those represented in FIG. 2A-B. The incorporation of the obstruction 202 ensures that administration of the drug or vaccine 2004 contained in the reservoir 203 is only possible once the hypodermic needle has been injected and the obstruction 202 has dissolved in the receiving living tissues. One preferred realisation of the present invention incorporates devices preloaded with solutions or suspensions of the drug or vaccine in non aqueous liquids, such as, and with out limitation, oils or fluorocarbons. Preferably the material that composes the obstruction 202 is not soluble in these non-aqueous liquids and therefore does not dissolve until the needle has penetrated the receiving living tissue.

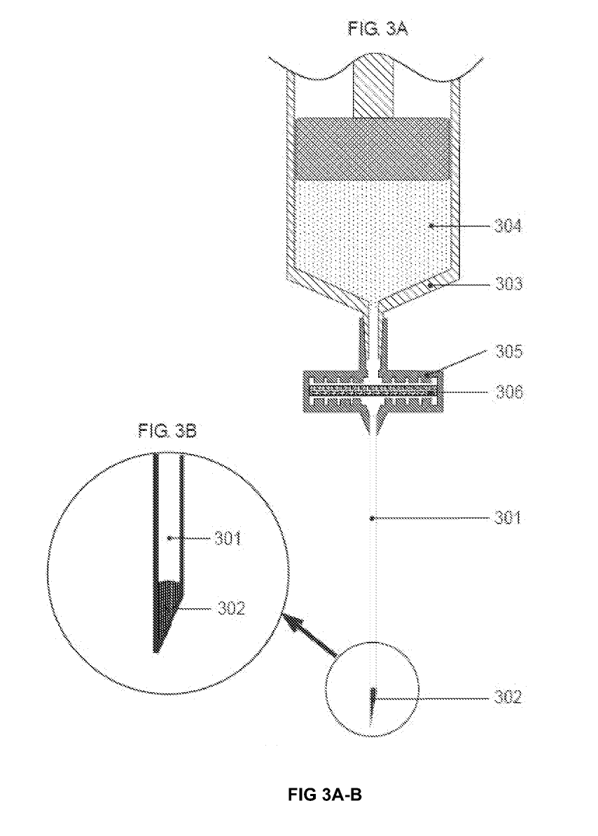

In another realisation represented in FIGS. 3A-B, the hypodermic needle 301 that incorporates at its sharp-end the obstruction 302, also incorporates a cartridge 305 that contains the drug or vaccine 306 stabilised in a solid format. Administration of the drug or vaccine involves the assembly with an injector device 303 containing saline or water for injection 304. Injection of the hypodermic needle into living tissues results in the dissolution of the obstruction 302 permitting the flow of the water for injection 304 through the cartridge, and carry over of the dissolved drug or vaccine 306.

The hypodermic needles describes in the present invention incorporate at their sharp-end a soluble obstruction 102, 202 or 302 which makes them functional only once they encounter an aqueous media such as a living tissue, as is illustrated, with out limitation, in the following three examples.

### Example 1

### Injection of Tetanus Vaccine Formulated as Suspension in Oil by Means of a Preloaded Device Incorporating a Hypodermic Needle with a Soluble Obstruction at its Sharp-End

The device represented in FIGS. 2A-B was made to incorporate a soluble obstruction 202 of trehalose at the sharp-end of a 0.6 mm internal diameter stainless steel bevelled hypodermic needle 201. For this, the sharp-end of the needle was dipped in a trehalose melt at about 100° C. Cooling of the tip resulted in the formation of an obstruction 202 of solid amorphous trehalose glass. The needle incorporating the obstruction was assembled to a flexible PVC ampoule as injector device 203. As a model drug or vaccine 204, the ampoule contained 1 ml of a suspension in sesame seed oil of 1-10 μm trehalose particles containing stabilised tetanus vaccine. Pressure of the final user on the pre-filled ampoule did not result in the ejection of any amount of the contained suspension. Intramuscular injection of the needle in a guinea pig resulted in the immediate dissolution of the obstruction 202 and permitted the administration of the complete dose of the vaccine suspension by gentle pressure by the user over the ampoule.

### Example 2

### Injection of a Hepatitis B Vaccine Stabilised in a Solid Format in a Cartridge Located at the Syringe End of a Hypodermic Needle Incorporating a Soluble Obstruction at its Sharp-End

The device represented in FIGS. 3A-B was manufactured to incorporate a soluble obstruction 303 of mannitol at the sharp end of a 0.3 mm internal diameter stainless steel bevelled hypodermic needle 201. For this the sharp-end tip of the hypodermic needle was introduced in a 60% mannitol solution in water at about 70° C. Removal of the needle and cooling of the tip resulted in the formation of a solid obstruction with a mannitol crystal at the sharp-end of the needle. The needle carried at the syringe end a cartridge 305 containing as a model drug or vaccine 306 a hepatitis B vaccine stabilised in an amorphous water-soluble trehalose glass. Prior to injection a conventional syringe 303 containing 1 ml of water for injection, was assembled to the hypodermic needle 301. Once assembled, pressure over the syringe 303 did not result in the ejection of any amount of liquid trough the syringe. Subcutaneous injection of the needle into a guinea pig and gentle pressure over the syringe resulted in the immediate dissolution of the obstruction 302 permitting the flow of water for injection 304 through the cartridge 305 and administration of a full dose of the drug 306.

### Example 3

### Evaluation of Suitability of Different Needle Obstruction Materials

A hypodermic bevelled stainless steel needle, as for example in 101, with an internal diameter of 0.8 mm was used to evaluate suitability of different materials to create the sharp-end obstruction 102. The needles containing approximately 1-3 mm³ volume of the different obstruction materials were assembled on to a syringe containing 1 ml sesame seed oil and injected into a set 3% gelatine block at 25° C. as a model of a living tissue 103. Dissolution time was estimated from the time of injection. Rapidly dissolving materials generally categorised as suitable needle-end obstruction materials (++++) while slower dissolving materials generally resulted in delayed injection and were categorised as less suitable obstructing materials (+).

| | Suitability as |
|---|---|
| | needle |
| | obstructing |
| Obstruction Material | material |
| Trehalose (from melt) | +++ |
| Trehalose + 2% sodium bicarbonate (from melt) | ++++ |
| Trehalose + 5% Calcium Lactate + 2% sodium | ++++ |
| bicarbonate | |
| Sucrose (from solution) | +++ |
| Sucrose + 5% Sodium Sulphate (from solution) | ++++ |
| Trehalose + 5% Calcium Lactate (from solution) | +++ |
| 20% Mannitol + 70% Trehalose (from melt) | ++++ |
| Mannitol (from melt) | ++++ |
| Hydroxyectoine (from solution) | ++++ |
| Glycine Betaine (from solution) | ++++ |
| Polyvinyl alcohol (from solution) | + |
| Proline | ++++ |
| Arginine•HCl | ++++ |
| Cysteine | + |
| Alanine | ++++ |
| Tyrosine | + |

1. A hypodermic needle comprising a sharp end and a water-soluble obstruction at said sharp end, wherein said water-soluble obstruction dissolves once the hypodermic needle is injected into human or animal tissues to allow passage of the liquid to be injected.

2. The hypodermic needle according to claim 1 wherein said obstruction comprises an agent selected from maltodextrin, trehalosa, raffinose cellobiose, melezitose, glucose, fructose, sucrose, maltulose, iso-maltulose, lactulose, maltose, gentobiose lactose, galactose, isomaltose, manose, maltitol, lactitol, erythritol, palatinitol, inositol, xilitol, mannitol, sorbitol, dulcitol and ribitol.

3. The hypodermic needle according to claim 2 wherein said agent is selected from the group consisting of trehalose, raffinose, sucrose, glucose and mannitol.

4. The hypodermic needle according to any of claim 1 wherein said obstruction comprises an agent selected from proline, cysteine, arginine, glutamine, glycine, glycine-betaine, ectoin and hydroxyectoine.

5. The hypodermic needle according to claim 1 wherein said obstruction comprises an agent selected from collagen, hyaluronic acid, fibronectin, gelatine, and agarose.

6. The hypodermic needle according to claim 1 wherein said obstruction comprises an agent selected from a polyvinyl alcohol, polyvinylpyrrolidone and a polyethylene glycol.

7. The hypodermic needle according to claim 1 wherein said obstruction comprises an agent that promotes an effervescence upon injection into said human or animal tissues.

8. The hypodermic needle according to claim 7 wherein said agent is a sodium carbonate.

9. The hypodermic needle according to claim 7 wherein said agent is a rapidly dissolving efflorescent salt.

10. The hypodermic needle according to claim 9 wherein said rapidly dissolving efflorescent salt is selected from the group consisting of sodium sulphate, acetate trihydrate, tetraborate decahydrate, bromoiridite dodecahydrate, carbonate heptahydrate metaperiodate trihydrate, metaphosphate hexahydrate, hydrogen orthophosphate dodecahydrate, sulphite heptahydrate, thiosulphate pentahydrate, calcium lactate, magnesium salicylate tetrahydrate, magnesium sulphate heptahydrate, and ammonium sulphate.

11. A method of improving a device designed for parenteral administration of a drug, vaccine, or another medical or veterinary product comprising incorporating the hypodermic needle according to claim 1, in said device.

12. The method of claim 11 wherein said drug, vaccine or other medical or veterinary product is formulated into a non-aqueous liquid.

13. The method of claim 11 wherein said drug, vaccine or other medical or veterinary product is contained in a solid format in a cartridge, filter or support.
